(19) United States
(12) Patent Application Publication
(10) Pub. No.: US 2016/0235456 A1
(43) Pub. Date: Aug. 18, 2016

(54) BIODEGRADABLE BONE NAIL

(71) Applicant: DONGGUAN EONTEC CO., LTD., Dongguan (CN)

(72) Inventors: Yonghua LAO, Dongguan (CN); Yangde LI, Dongguan (CN); Weirong LI, Dongguan (CN); Fangfei LIU, Dongguan (CN)

(21) Appl. No.: 15/095,148

(22) Filed: Apr. 10, 2016

Related U.S. Application Data
(63) Continuation-in-part of application No. PCT/CN2014/083364, filed on Jul. 31, 2014.

(30) Foreign Application Priority Data
Oct. 11, 2013 (CN) 201320625541.4

(51) Int. Cl. A61B 17/86

(52) U.S. Cl. CPC A61B 17/864; A61B 17/866; A61B 2017/00004

(57) ABSTRACT

A biodegradable bone nail, including a rod-shaped magnesium screw having a head including a cross groove. The magnesium screw includes an axial chamber disposed below the cross groove, and a depth of the axial chamber is two thirds that of the bone nail. Along the length direction of the axial chamber, a plurality of radial holes are equidistantly disposed every 90 degrees from the bottoms of threads on the wall of the magnesium screw, and the radial holes communicate with the axial chamber.

### CROSS-REFERENCE TO RELATED APPLICATIONS

This application is a continuation-in-part of International Patent Application No. PCT/CN2014/083364 with an international filing date of Jul. 31, 2014, designating the United States, now pending, and further claims priority benefits to Chinese Patent Application No. 201320625541.4 filed Oct. 11, 2013. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### BACKGROUND OF THE INVENTION

1. Field of the Invention

The invention relates to an orthopedic device and in particular to a biodegradable bone nail.

2. Description of the Related Art

Conventional bone nails are made of stainless steel, titanium alloy, or magnesium alloy. The former two are not degraded in the body and may need to be removed after fracture healing. This increases pain and cost, and can lead to complications. Magnesium alloy is an absorbable implant material, but the components thereof are complex, often resulting in cytotoxicity and adverse biological reactions.

### SUMMARY OF THE INVENTION

In view of the above-described problems, it is one objective of the invention to provide a biodegradable bone nail that has simple structure and causes no cytotoxicity and no adverse biological reactions.

To achieve the above objective, in accordance with one embodiment of the invention, there is provided a biodegradable bone nail, comprising a rod-shaped magnesium screw having a head comprising a cross groove, the magnesium screw comprising an axial chamber disposed below the cross groove, a depth of the axial chamber being two thirds that of the bone nail. Along a length direction of the axial chamber, a plurality of radial holes are equidistantly disposed every 90 degrees from bottoms of threads on a wall of the magnesium screw, and the radial holes communicate with the axial chamber.

In a class of this embodiment, the magnesium screw has a magnesium purity of no less than 99.9%.

Advantages of the biodegradable bone nail according to embodiments of the invention are summarized as follows. The biodegradable bone nail is made of pure magnesium having a purity of between 99.9% and 99.99%. As a fixed implantable apparatus for bone repair and reconstruction, the biodegradable bone nail causes no cytotoxicity and no adverse biological reactions. When the biodegradable bone nail is used for bone repair and reconstruction, prior to implanting, the axial chamber of the bone nail is filled with patient's own bone flap or cortical bone. The bone flap or cortical bone is compacted. The compacted bone flap or cortical bone significantly enhances the torsional strength of the bone nail and ensure the uniform stress of the bone nail during being implanted. In addition, patient's own bone flap or cortical bone filled in the axial chamber communicates with the bone tissues outside the bone nail through the radial holes to induce the growth of the bone, thus facilitating bone healing.

### BRIEF DESCRIPTION OF THE DRAWINGS

The invention is described hereinbelow with reference to the accompanying drawings, in which:

FIG. 1 is a schematic diagram of a biodegradable bone nail in accordance with one embodiment of the invention; and

FIG. 2 is a sectional view of a biodegradable bone nail in accordance with one embodiment of the invention.

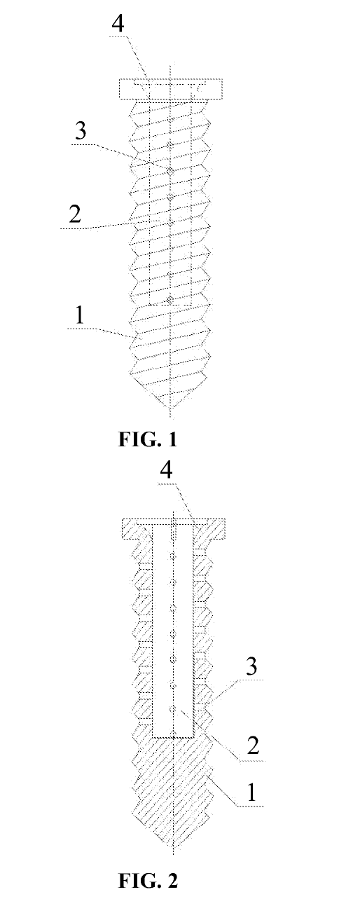

In the drawings, the following number references are used: 1. Bone nail; 2. Axial chamber; 3. Radial hole; 4. Cross groove.

### DETAILED DESCRIPTION OF THE EMBODIMENTS

For further illustrating the invention, experiments detailing a biodegradable bone nail are described below. It should be noted that the following examples are intended to describe and not to limit the invention.

A biodegradable bone nail 1 comprises a rod-shaped magnesium screw having a head comprising a cross groove 4. The magnesium screw comprises an axial chamber 2 disposed below the cross groove 4, a depth of the axial chamber being two thirds that of the bone nail. Along a length direction of the axial chamber, a plurality of radial holes 3 are equidistantly disposed every 90 degrees from bottoms of threads on a wall of the magnesium screw, and the radial holes 3 communicate with the axial chamber 2.

The biodegradable bone nail is made of pure magnesium having a purity of between 99.9% and 99.99%. As a fixed implantable apparatus for bone repair and reconstruction, the biodegradable bone nail causes no cytotoxicity and no adverse biological reactions. When the biodegradable bone nail is used for bone repair and reconstruction, prior to implanting, the axial chamber 2 of the bone nail 1 is filled with patient's own bone flap or cortical bone. The bone flap or cortical bone is compacted. The compacted bone flap or cortical bone can significantly enhance the torsional strength of the bone nail 1 and ensure the uniform stress of the bone nail 1 during implantation. In addition, the bone flap or cortical bone filled in the axial chamber 2 can communicate with the bone tissues outside the bone nail through the radial holes 3 to induce the growth of the bone, thus facilitating the bone healing.

Unless otherwise indicated, the numerical ranges involved in the invention include the end values. While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A biodegradable bone nail, comprising a rod-shaped magnesium screw having a head comprising a cross groove, the magnesium screw comprising an axial chamber disposed below the cross groove, a depth of the axial chamber being two thirds that of the bone nail, wherein, along a length direction of the axial chamber, a plurality of radial holes are equidistantly disposed every 90 degrees from bottoms of threads on a wall of the magnesium screw, and the radial holes communicate with the axial chamber.

2. The bone nail of claim 1, wherein the magnesium screw consists of magnesium having a purity of no less than 99.9%, the balance being impurities.
